(19) United States
(12) Patent Application Publication
(10) Pub. No.: US 2006/0195189 A1
(43) Pub. Date: Aug. 31, 2006

(54) Prosthetic joint of cervical intervertebral for a cervical spine

(75) Inventors: Helmut D. Link, Hamburg (DE); Arnold Keller, Kayhude (DE); Paul C. McAfee, Baltimore, MD (US)

Correspondence Address: MORRISON & FOERSTER LLP, 1650 TYSONS BOULEVARD, SUITE 300, MCLEAN, VA, 22102, US

(73) Assignee: Cervitech, Inc., Rockaway, NJ (US)

(21) Appl. No.: 10/552,707

(22) PCT Filed: Feb. 4, 2004
(86) PCT No.: PCT/EP04/01029

§ 371 Date: Oct. 7, 2005

(30) Foreign Application Priority Data
Apr. 7, 2003 (EP) 03008127.7

(51) Int. Cl. A61F 2/44; A61B 17/16

(52) U.S. Cl. 623/17.11; 606/85

(57) ABSTRACT

In an intervertebral joint prosthesis for an intervertebral space of the cervical spine, the intervertebral space is delimited by the end plates of the adjacent vertebral bodies. The bearing surfaces of these end plates, when viewed in a frontal plane, have edge zones laterally adjacent to a substantially flat central area that are more strongly curved than the flat central area. These edge zones are also more strongly mineralized than the central area and are therefore particularly stable. At least one of the prosthesis surfaces intended to bear on a vertebral body surface has a lateral extend reaching into the edge zones. The convex curvature of this prosthesis surface, when viewed in the frontal plane, is at least as great as the corresponding curvature of the surface of the end plates. This ensures that the prosthesis is also supported on the particularly stable edge zones, and these edge zones do not have to be subjected to any substantial removal of material.

Joint prostheses for replacement of an intervertebral disk of the cervical spine are known which are composed of two cover plates and a hinge core. The cover plates, arranged approximately parallel to one another on both sides of the core, have surfaces intended for connection to the end plates of the adjacent vertebral bodies. Known prostheses of this type (FR-A-2718635, EP-B-699426, WO 03063727, WO 0211650, EP-A-1166725, EP-A-820740) are circularly delimited. Since the end plates of the vertebral bodies are considerably wider than deep in the AP direction, these known prostheses do not exploit the extent of the naturally available surfaces for force transmission. As a consequence of this, greater forces arise between the prosthesis surfaces and the vertebral bodies than would be the case if the surfaces were better utilized. In intervertebral disk prostheses intended for the lumbar spine, the best utilization of space is achieved by using an oval prosthesis contour (WO 0101893, EP-B-471821, EP-A-747025) or kidney-shaped configuration (EP-A-747025). Rectangular prosthesis shapes are also known (U.S. Pat. No. 5,425,773).

Inventions for which applications have previously been filed by the same Applicant or its legal predecessors (EP-A-1344508, EP-A-1344507, WO 03075803, WO 03075804) disclose a prosthesis contour shape which is approximate to a rectangle with rounded corners and covers the substantially flat area of the end plates of the vertebral bodies. They achieve a much better utilization of space and more reliable long-term connection to the vertebral bodies than do circularly delimited prostheses. In addition, they have a low height and therefore require only a small amount of natural bone substance to be removed for preparing the implantation space. In many cases, they permit complete or partial preservation of the hard but, in the case of the cervical vertebrae, very thin cortical bone.

Unlike cervical joint prostheses, cages are used for immovably fixed connection of adjacent vertebral bodies for the purposes of their fusion. Since they are intended for union of the vertebrae, less importance is placed on the quality of their actual long-term connection to the bone. The preservation of the natural bone substance is also less important since it is replaced by homologous material stored in the cage (EP-B-179695, WO 9720526, U.S. 2001/0016774, WO 0191686, WO 9000037).

The invention aims to develop the prosthesis type (WO 03075804) disclosed in the aforementioned earlier applications, with the objective of improved force transmission between the prosthesis and the end plates of the vertebral bodies, while at the same time substantially preserving the natural bone substance.

The invention is based on the knowledge that the end plates of the vertebral bodies of the cervical spine have a different degree of mineralization in different regions. The greater the mineralization, the more compact the bone substance and the more suitable it is to take up forces. It has been found that the highest degree of mineralization is present in lateral edge zones of the end plates of the vertebral bodies where the substantially flat central area of these end plates, in frontal section, merges into a stronger curvature that leads to the uncovertebral joints. The underlying concept of the invention lies in using these edge zones for transmission of forces between the prosthesis and the bone. The prosthesis surfaces intended to bear on the vertebral body surface are extended laterally into the more strongly mineralized and at least partially more strongly curved lateral edge zones of the vertebral body surface. So that the greater strength of these edge zones of the end plates of the vertebral bodies can be utilized, they must be preserved even if the prosthesis height or the adaptation of the bone to the prosthesis shape demands a certain degree of milling of the end plates of the vertebral bodies. According to the invention, this milling is limited substantially to the central area of the end plates of the vertebral bodies where the bone strength is lesser anyway, whereas the stronger edge zones are completely or partially preserved. The prosthesis shape according to the invention permits this by virtue of the extent of its convex curvature. This curvature is chosen to be at least as great as the opposite curvature of the associated end plate surface. It is generally greater. That is to say the central areas of the prosthesis surface protrude farther upward or downward than the edge zones in relation to the surface of the vertebral bodies. The height of the prosthesis is limited in the edge zones such that milling of the bone there can be omitted. Only the cartilage is removed and, if appropriate, the bone surface is trimmed a little for the purpose of better connection to the prosthesis. If milling is in fact necessary, it can be limited mainly to the central area. The shape relationships according to the invention can also be defined in that the prosthesis surface is similar to and complements the shape of the end plates of the vertebral bodies in frontal section but protrudes farther in the central area, relative to the average shape of the end plates. A further alternative characterization of the prosthesis shape is that the height of the prosthesis in the caudocranial direction in the lateral edge areas is approximately equal to the height of a average intervertebral space taken as a standard, whereas it is greater in the central area. The dimensions are chosen such that, when used in an averagely shaped intervertebral space, slight milling is carried out in the central area but not in the edge zones of the front section in question. In many cases, milling of the central area can also be dispensed with.

The greater compliance of the bone substance in the central area, irrespective of whether it is milled or not, establishes a good condition for a form-fit connection to the prosthesis surface when the latter is provided with suitable elevations and depressions, which are designed in particular as teeth. It can also be provided with a coating that promotes connection to the bone.

The more strongly mineralized edge zones of the end plates of the vertebral bodies are inclined in the frontal section as a transition to the uncovertebral joints. A corresponding inclination is expediently also present on those edge zones of the prosthesis surface which are intended to bear thereon. On the underside of the prosthesis, the angle of inclination relative to the main direction of extent of the prosthesis is expediently 20°. On the top of the prosthesis, this inclination is expediently at least 0° and preferably 10 to 30°.

So that the prosthesis surface reaches the more strongly mineralized edge zones of the end plates of the vertebral bodies, the width of the prostheses should be chosen to be at least 1.5 times as great as the depth by which they are intended to lie in the intervertebral space in the anteroposterior direction. This factor is preferably greater than 1.63.

It is not necessary for the above-indicated shape characteristics to apply to the entire depth of the prosthesis. Although this is certainly possible, it is nevertheless more expedient, in many cases, if only the dorsal half of the prosthesis is configured according to the invention. This is due to the fact that the greatest degree of mineralization of the end plates of the vertebral bodies is reached in the dorsolateral corner areas thereof.

The invention is explained in more detail below with reference to the drawing which shows advantageous illustrative embodiments of the invention. In said drawing:

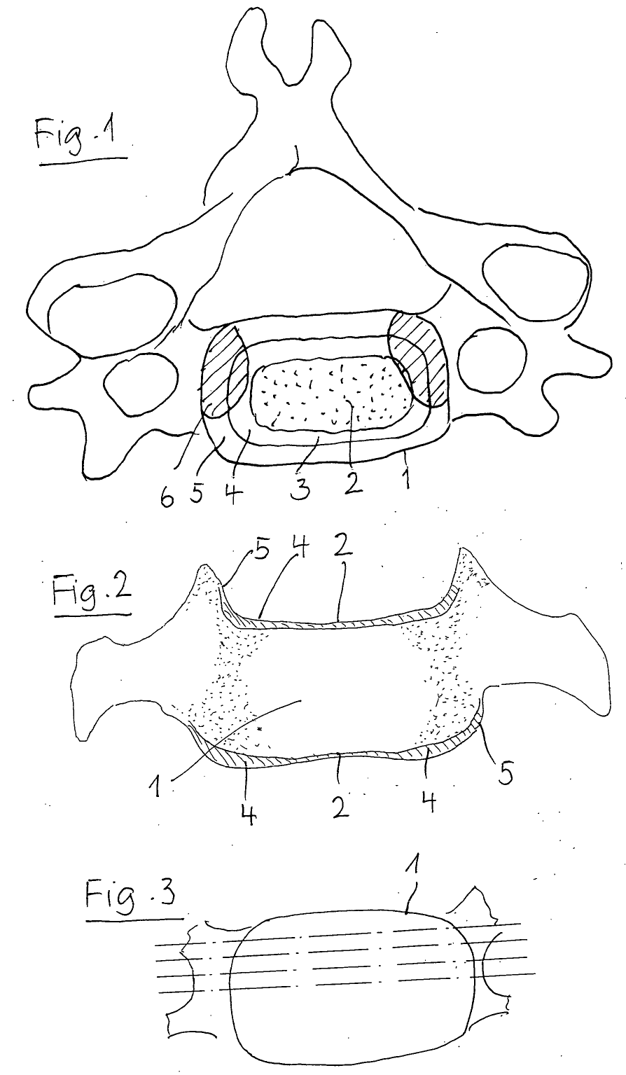

FIG. 1 shows a plan view of a cervical vertebra,

FIG. 2 shows a frontal section through the vertebral body in accordance with one of the dot-and-dash lines in FIG. 3,

FIG. 3 shows a plan view of a vertebral body with indicated frontal planes,

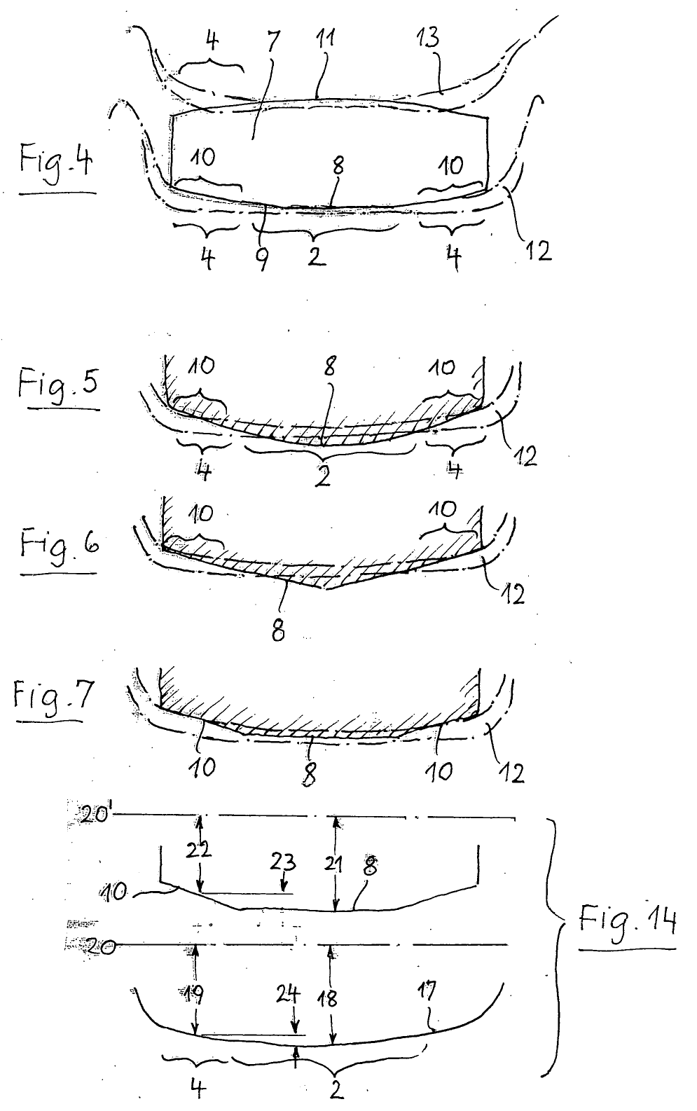

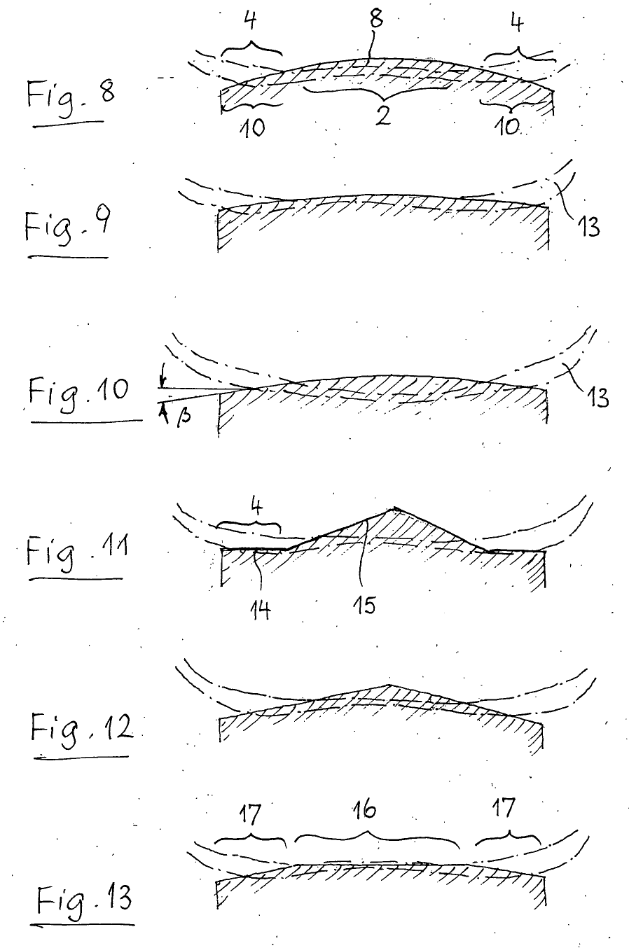

FIG. 4 shows the outline of a prosthesis within a frontal plane according to FIG. 3,

FIGS. 5 to 7 show different caudal prosthesis contours in comparison with the contours of the associated end plate of a lower vertebral body in the frontal section,

FIGS. 8 to 13 show different cranial prosthesis contours in comparison with the contours of the associated end plate of an upper vertebral body in the frontal section,

FIG. 14 shows a view illustrating the height differences of prostheses and end plate surfaces,

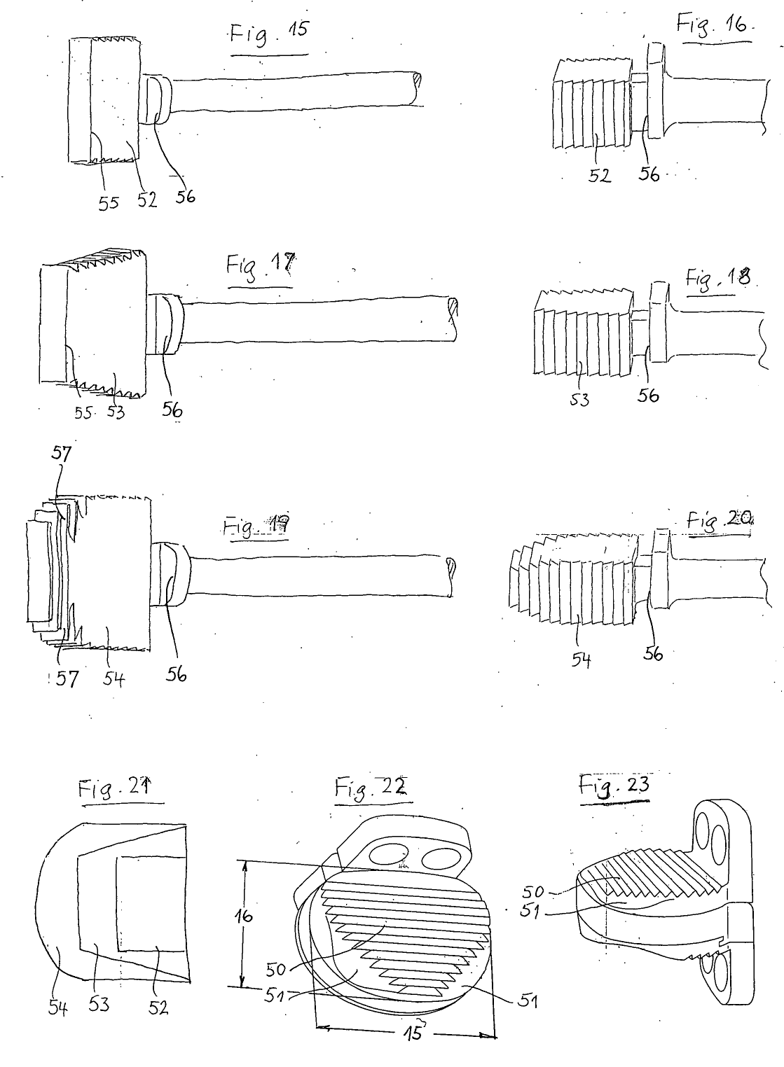

FIGS. 15 to 20 show three rasps for preparing the insertion space for the prosthesis,

FIG. 21 shows the outline of the three rasps for comparison purposes, and

FIGS. 22 and 23 show perspective views of a prosthesis from different directions.

If one considers the upper end plate of a vertebral body 1, it is found that it is thin and porous in a central area 2. This is surrounded by an edge zone 3 which is more strongly mineralized, has minimal porosity and is substantially thicker than the end plate in the central area 2. The lateral portions 4 of this edge zone 3 ascend to the steep flanks 5 of the uncovertebral joints. The same situation is repeated on the underside of the vertebral body with the reverse direction of curvature. It has been found that a particularly high degree of mineralization is present in the edge zones 4 and the flanks 5, specifically in the dorsolateral areas 6, which are indicated in FIG. 1 by hatching. The more strongly mineralized areas have a greater load-bearing capacity and are also better supported by the underlying spongy bone tissue, as is indicated by stippling. In many cases, the lateral edge zones 4 merge with a continuously increasing inclination into the flanks 5, without an anatomical border being clearly recognizable. However, a border is shown in FIG. 1 to permit better understanding. This is the line below which the lateral zones 4 lie which are used for supporting the prosthesis in the manner according to the invention, whereas the flanks 5 lying above said line are too steep for this purpose, that is to say steeper than a desired limit value, which generally lies between an inclination of 20 and 40°.

This supporting of the prosthesis in the lateral edge zones 4 is made clear in FIG. 4, which shows a cross section along one of the frontal planes indicated by dot-and-dash lines in FIG. 3. The prosthesis is shown by solid lines, and the end plates of the vertebral bodies are shown by dot-and-dash lines. Assumed is a prosthesis 7 whose bottom surface 9 has an approximately flat central area 8, cooperating with the central area 2 of the upper end plate 12 of the lower vertebral body, and zones 10 which are beveled to the sides and which cooperate with the lateral edge zones 4 of the lower vertebral body. In the cross section illustrated, the prosthesis shape approximately corresponds to the shape of the upper end plate of the lower vertebral body, so that no milling, or only slight milling, of the vertebral body is needed to adapt it to the prosthesis. It is desirable that, in the edge zones 4 of the end plate, only the cartilage resting on the bone substance is removed, whereas the bone substance itself is left intact or is just trimmed a little so as to adapt adequately to the prosthesis shape and be better connected thereto.

In the example shown in FIG. 4, milling is likewise not particularly required in the central area 8 of the bottom surface of the prosthesis. However, at least a trimming of the bone is desired here so that the latter connects better to the central prosthesis surface 8. To make this easier, the prosthesis surface, in its central area 8, is configured so as to permit an intimate and permanently fixed connection to the bone. It can in particular be provided with elevations and depressions (see the teeth in FIGS. 21, 22) and with a coating that activates the growth of bone.

Other prosthesis shapes complying with this underlying concept of the invention are shown in FIGS. 5 to 7. FIG. 5 shows, in frontal section, a uniformly rounded prosthesis bottom surface which requires virtually no milling of bone in the edge zones 4, whereas the central area 2 is milled more deeply. Instead of the deeper milling of the central area, provision can also be made for the central area 8 of the prosthesis surface to be configured in such a way that, without any milling of bone, or after slight milling of bone, it sinks elastically into the remaining bone substance. The same applies to the prosthesis shapes which are shown, in FIG. 6, as an inverted roof shape and, in FIG. 7, with a central flat area and rising edge zones 10 (similar to the embodiment according to FIG. 4).

As regards the shape of the top 11 of the prosthesis in the example in FIG. 4, it likewise applies that the lateral edge zones 4 of the associated end plate 13 of the vertebral body can be substantially preserved, whereas slight milling is required in the central area. The lateral edge zones 4 of the end plate of the vertebral body can therefore play an active part in the transmission of forces. An important part of the force transmission also takes place in the central area. However, this area, by virtue of its intimate toothed connection to the prosthesis surface, also serves for the long-term anchoring of the prosthesis in the intervertebral space.

The shape examples shown in FIGS. 8 and 9 show, in frontal section, dome-shaped prosthesis surfaces 8 of different degrees of curvature. It is assumed here that the associated end plate 13, in frontal section, is of a slightly concave configuration, that is to say of a complementary configuration. By contrast, FIG. 10 assumes a weakly convex end plate 13 and shows that, in this case too, the lateral edge zones of the end plate 13 can be substantially preserved and the milling of the bone is limited to the central area. FIG. 11 shows an example in which the top of the prosthesis is almost flat in the edge zones 14, so as to be able to be better applied to the edge zones 4 of the bone, whereas the central area 15 has a cone shape or roof shape. Secure positioning of the prosthesis on the bone is achieved in this way. Moreover, this central area, in the same way as in the other embodiments, can be provided with a small area of toothed engagement with the bone substance. In the illustrative embodiment according to FIG. 12, the entire top of the prosthesis is roof-shaped or cone-shaped. This also saves the lateral edge zone of the bone and limits any milling to the central area. Finally, FIG. 13 shows a prosthesis top which is flat in the central area 16 and beveled in the lateral edge areas 17. This shape is especially advantageous because very slight milling of the bone is sufficient not only in the lateral edge zones but also in the central area.

In all the examples, the top and bottom faces of the prosthesis are of a convex design. To put it another way, the prosthesis has a greater height in the central area than in its edge areas. This is favorable for the accommodation of a lens-shaped prosthesis core (see, for example, WO 03/075804). By contrast, the prosthesis core requires less height in the edge zones. In this way, the overall height of the prosthesis can be kept low. In particular, it can be kept so low that milling in the lateral edge zones of the end plates of the vertebral bodies can generally be dispensed with.

To ensure that the edge zones 10 of the lower prosthesis surface are able to cooperate with the edge zones 4 of the surface of the end plates, they must be inclined approximately the same as these. This inclination α (FIG. 5) is defined in relation to the main plane 14 of the prosthesis or of the intervertebral space and should be at least 20° in the lower prosthesis surface. It is preferably of the order of 30° or over. The farther the prosthesis extends laterally into the edge zone with the greater degree of mineralization, the greater the angle of inclination it must reach.

The corresponding angle β (FIG. 10) on the top of the prosthesis can be shallower because the prosthesis there is not confined by the ascending flank of the uncovertebral joint. It can be as little as 0° and is preferably 10 to 30°.

The preferred height relationships of the prosthesis with respect to the associated end plate can be inferred from FIG. 14. In relation to an imaginary midplane 20 (or any other desired plane parallel thereto), the end plate surface 17 has, in its central area 2, a height designated by the arrow 18, and, in its lateral edge zone 4, a mean height according to the arrow 19. The heights of the prosthesis surface relative to a corresponding midplane 20′ are indicated by the arrows 21 and 22. According to the invention, the difference 23 between the heights 21 and 22 of the prosthesis should be at least equally as great as the difference 24 between the heights 18 and 19 of the end plate surface. If this condition is satisfied, it is possible to achieve the objective that the lateral edge zones 4 of the end plate, compared to the latter's central area, do not have to be subjected to so much removal of material. This correspondingly also applies to the top of the prosthesis.

When, in this description, predetermined shapes and dimensions of the vertebral bodies and of their end plates have been assumed, this always means that standardized shapes and dimensions are intended which have been obtained from a large number of measurements on natural vertebral bodies and have been standardized so as to form a basis from which suitable prosthesis shapes and dimensions can be found. A supplier of cervical intervertebral prostheses will normally provide a large number of prostheses having different shapes or dimensions, so that the physician can select the most suitable one for the particular application.

In connection with the invention, only the shape of the prosthesis in the frontal section has been dealt with. In the sagittal section, the prosthesis can be of any desired shape. For example, its top and bottom surfaces can be substantially straight or curved in a central sagittal section.

To ensure that the bone surfaces obtain exactly the shape needed for application of the invention, a set of rasps is provided. These are shown in FIGS. 15 to 21. They are configured such that they prepare the surface shape of the vertebrae for receiving the prosthesis. The examples shown are directed at the illustrative embodiment of the prosthesis shown in FIGS. 22 and 23. It has a rectangular contour with rounded corners which is suitable for substantial utilization of the extent of the intervertebral space, including the lateral edge zones. It is so flat that it can be inserted without deep milling of the end plates of the vertebral bodies. Facing the vertebral bodies, it has outer surfaces which, across their largest part 50, are approximately flat and toothed. Its dorsolateral corners 51 are beveled in such a way that, in this area, frontal sections according to FIG. 3 have approximately the contour shape of the prosthesis shown in FIGS. 7 and 13.

The contour shape is prepared in the intervertebral space by using the set of rasps 52, 53 and 54 shown in FIGS. 15 to 20. The graded sizes of the rasps are shown in FIG. 21. After the vertebral bodies involved have been adjusted by instruments (not shown) to the spacing they are intended to have after insertion of the prosthesis, the smallest rasp 52 is pushed into the intervertebral space by way of a handle (not detailed) in order to open up the access. Its depth of insertion is limited by the stop 56. Accordingly, it passes into the intervertebral space no deeper than is shown in FIG. 21. It is followed by rasp 53 which has a trapezoid shape approximately corresponding to the trapezoid shape of the flat surface part 50 of the prosthesis surface. Finally, the rasp 54 shapes the intervertebral space so that it substantially coincides with the shape of the prosthesis that is to be fitted. The height of the rasps is equal to that of the prosthesis.

The rasps are not toothed on those surfaces which correspond to the flat part 50 of the prosthesis. This means that they effect only a slight abrasion with their front edge 55. If, by contrast, the prosthesis is configured such that it requires greater milling of the vertebral bodies in the central area, these surfaces of the rasp can also be provided with teeth. In the areas 57 of the rasp 54 which are assigned to the dorsolateral areas of the edge zones of the end plates of the vertebral bodies, teeth are provided in order to free cartilage from the areas of the lateral edge zones in question and, if appropriate, to adapt them to the prosthesis shape.

Once the end plates of the vertebral bodies have been trimmed in their central area for receiving the toothed, central area 50 of the prosthesis, the prosthesis tips sink into the relatively compliant surface of the bone until the beveled edge zones 51 of the prosthesis bear on the lateral edge zones 4 of the end plates of the vertebral bodies.

1. (canceled)

2. The prosthesis as claimed in claim 11, having a height in a caudo-cranial direction relative to an orientation of the prosthesis in an implanted position in portions of the prosthesis configured to engage the lateral edge zones approximately equal to a height of the intervertebral space at the location of the edge zones, and having a height in portions of the prosthesis configured to engage the central area greater than a height of the intervertebral space at the location of the central area.

3. The prosthesis as claimed in claim 11 or 2, wherein the prosthesis surface is provided with elevations and depressions in the central area but not in the edge area.

4. The prosthesis as claimed in claim 11 or 2, wherein the prosthesis surface is toothed in the central area.

5. The prosthesis as claimed in claim 11 or 2, wherein an angle of inclination of the portion of a lower prosthesis surface that is configured to engage the edge zones of the end plate surfaces in the frontal plane relative to the main direction of extent of the prosthesis relative to an orientation of the prosthesis in an implanted position is at least 20°.

6. The prosthesis as claimed in claim 11 or 2, wherein an angle of inclination of the portion of an upper prosthesis surface that is configured to engage the edge zones of the end plate surfaces relative to the main direction of extent of the prosthesis relative to an orientation of the prosthesis in an implanted position is at least 0°.

7. The prosthesis as claimed in claim 11 or 2, wherein the prosthesis has a width that is at least 1.5 times as great as its depth in the intervertebral space relative to an orientation of the prosthesis in an implanted position.

8. The prosthesis as claimed in claim 11 or 2, wherein the specified shape of the prosthesis is limited to its dorsal half.

9. The intervertebral joint prosthesis as claimed in claim 11 or 2, wherein the surface of at least one of its cover plates, whose size is dimensioned to substantially utilize the naturally provided surface extent of the intervertebral space, has a central area (8, 50), which extends approximately parallel to the main plane of extent of the cover plate, and, adjoining this in the dorsolateral direction, a surface (10, 51) beveled relative to the central area.

10. An instrument set configured for inserting the prosthesis as claimed in claim 11 or 2, comprising a plurality of rasps adapted to the configuration of the prosthesis and configured to prepare the vertebral body surfaces to accommodate the prosthesis shape,
the rasps being designed such that the rasps remove material from the central area and the edge zones except for the dorsal part of the edge zones.

11. An intervertebral joint prosthesis configured for implantation into an intervertebral space between adjacent vertebral bodies of the cervical spine, which intervertebral space is delimited by end plates of the adjacent vertebral bodies whose end plate surfaces whose surfaces laterally adjacent to a substantially flat central area include edge zones that are more strongly curved than the substantially flat central area,
at least one of the prosthesis surfaces being configured to bear on a vertebral body surface having a lateral extend reaching into the edge zones, the convex curvature of this prosthesis surface in a frontal plane being at least as great as the corresponding curvature of the end plate surfaces.

12. The prosthesis as claimed in claim 6, wherein the angle of inclination of the portion of the upper prosthesis surface that is configured to engage the edge zones of the end plate surfaces relative to the main direction of extent of the prosthesis relative to an orientation of the prosthesis in an implanted position is 10 to 30°.
